(19) United States
(12) Patent Application Publication
(10) Pub. No.: US 2017/0055920 A1
(43) Pub. Date: Mar. 2, 2017

(54) GENERATING A RESPIRATION GATING SIGNAL FROM A VIDEO

(71) Applicant: Xerox Corporation, Norwalk, CT (US)

(72) Inventors: Lalit Keshav MESTHA, Fairport, NY (US); Sanjay BHARADWAJ, Mysore (IN); Prathosh A. PRASAD, Mysore (IN); Tejaskumar D. BENGALI, Mysore (IN); Himanshu J. MADHU, Mumbai (IN); Suresh KUPPUSWAMY, Bangalore (IN)

(21) Appl. No.: 14/837,898

(22) Filed: Aug. 27, 2015

(51) Int. Cl. A61B 5/00; A61B 5/113; A61B 5/11

(52) U.S. Cl. CPC A61B 5/7285; A61B 5/0077; A61B 5/0075; A61B 5/4836; A61B 5/1128; A61B 5/113; A61B 5/725; A61B 5/7264; A61B 5/0013; A61B 2090/374

(57) ABSTRACT

What is disclosed is a system and method for generating a respiration gating signal from a video of a subject for gating diagnostic imaging and therapeutic delivery applications which require respiration phase and/or respiration amplitude gating. One embodiment involves receiving a video of a subject and generating a plurality of time-series signals from the video image frames. A set of features are extracted from the time-series signals and multi-dimensional feature vectors are formed. The feature vectors are clustered. Time-series signals corresponding in each of the clusters are averaged in a temporal direction to obtain a representative signal for each cluster. One cluster is selected and a respiration gating signal is generated from that cluster's representative signal. Thereafter, the respiration gating signal is used to gate diagnostic imaging and therapeutic delivery applications which requires gating based on a threshold set with respect to either respiration phase or respiration amplitude.

### TECHNICAL FIELD

The present invention is directed to systems and methods for generating a respiration gating signal from a video of a subject for gating diagnostic imaging and therapeutic delivery applications which require respiration phase and/or respiration amplitude gating.

### BACKGROUND

In order to limit patient motion induced image degradation, it is preferable that data acquisition is gated (i.e. triggered) to coincide with that motion. This approach is termed prospective gating. If the motion of the patient is due to respiration then it is called prospective respiration gating. In contrast, if the motion is due to cardiac function then the gating is called prospective cardiac gating. Typically when the patient is moving, data acquisition is paused or is otherwise compensate for. Conventional systems acquire a gating signal using leads attached to the patient's body. Other systems use specialized belts that employ sensors which generate a signal used for gating purposes. Probes and belts may obscure the anatomical region being imaged. Moreover, probes, belts, wires, sensors, and the like may lead to patient discomfort. Significant advantages can be gained if a respiration gating signal can be obtained without patient contact using a video. The present invention is specifically directed to this end.

Accordingly, what is needed in this art are sophisticated systems and methods for generating a respiration gating signal from a video of a subject for gating diagnostic imaging and therapeutic delivery applications which require respiration phase and/or respiration amplitude gating.

### INCORPORATED REFERENCES

The following U.S. Patents, U.S. Patent Applications, and Publications are incorporated herein in their entirety by reference.

“Determining A Respiratory Pattern From A Video Of A Subject”, U.S. patent application Ser. No. 14/742,233, by Prathosh A. Prasad et al.

“Breathing Pattern Identification For Respiratory Function Assessment”, U.S. patent application Ser. No. 14/044,043, by Lalit K. Mestha et al.

“Processing A Video For Respiration Rate Estimation”, U.S. patent application Ser. No. 13/529,648, Lalit K. Mestha et al.

“Processing a Video for Tidal Chest Volume Estimation”, U.S. patent application Ser. No. 13/486,637, Edgar Bernal et al.

“Real-Time Video Processing For Respiratory Function Analysis”, U.S. patent application Ser. No. 14/195,111, Survi Kyal et al.

“System And Method For Determining Respiration Rate From A Video”, U.S. patent application Ser. No. 14/519,641, by Lalit K. Mestha et al.

“Removing Environment Factors From Signals Generated From Video Images Captured For Biomedical Measurements”, U.S. patent application Ser. No. 13/401,207, by Lalit K. Mestha et al.

“Minute Ventilation Estimation Based On Chest Volume”, U.S. patent application Ser. No. 13/486,715, by Edgar Bernal et al.

“Minute Ventilation Estimation Based On Depth Maps”, U.S. Pat. No. 8,971,985

“Respiratory Function Estimation From A 2D Monocular Video”, U.S. Pat. No. 8,792,969

“Monitoring Respiration With A Thermal Imaging System”, U.S. Pat. No. 8,790,269

### BRIEF SUMMARY

What is disclosed is a system and method for generating a respiration gating signal from a video of a subject for gating diagnostic imaging and therapeutic delivery applications which require respiration phase and/or respiration amplitude gating. One embodiment hereof involves the following. First, a video of a subject is received. The video comprises N≧2 image frames of a region of interest of the subject where a signal corresponding to the subject's respiratory function can be registered by at least one imaging channel of a video imaging device used to capture the video. The region of interest comprises at least P pixels, where P≧2. Next, a plurality of time-series signals {S₁, . . . , S_{P}} are generated, each of duration N whose samples are values of pixels in the region of interest in the image frames. For each of the time-series signals, a set of features are extracted and P-number of M-dimensional feature vectors are formed, where M≧2. The feature vectors are then clustered into K≧2 clusters. All time-series signals corresponding to pixels represented by the feature vectors in each of the clusters are then averaged in a temporal direction to obtain a representative signal for each cluster. One of the clusters is selected using, for example, a distance metric, and a respiration gating signal is generated from either the selected cluster's representative signal or from a respiratory pattern associated with the selected cluster's representative signal. Thereafter, the generated respiration gating signal is used to gate a device which requires gating based on a threshold set with respect to respiration phase or amplitude. The teachings hereof find their uses in a wide array of diagnostic imaging and therapeutic delivery applications such as, for instance, Dual Energy Radiography, Computed Tomography (CT), Tomographic Synthesis in Mammography, Magnetic Resonance Imaging (MRI), Positron Emission Tomography (PET), PET-CT, and PET-MRI.

Features and advantages of the above-described embodiments will become apparent from the following description and accompanying drawings.

### BRIEF DESCRIPTION OF THE DRAWINGS

The foregoing and other features and advantages of the subject matter disclosed herein will be made apparent from the following detailed description taken in conjunction with the accompanying drawings, in which:

FIG. 1 shows a frontal view and a rear view of an adult human subject;

FIG. 2 shows an example video imaging device capturing image frames of a region of interest of the subject of FIG. 1;

FIG. 3 is a flow diagram which illustrates one example embodiment of the present method for generating a respiration gating signal from a video of a subject;

FIG. 4 is a continuation of the flow diagram of FIG. 3 with flow processing continuing with respect to node A;

FIG. 5 is a continuation of the flow diagram of FIG. 4 with flow processing continuing with respect to node C;

FIG. 6 shows an example respiration gating signal with a threshold set with respect to respiration amplitude; and

FIG. 7 is a block diagram of one example video processing system 700 for processing a video in accordance with the embodiments described with respect to the flow diagrams of FIGS. 3-5.

### DETAILED DESCRIPTION

What is disclosed is a system and method for generating a respiration gating signal from a video of a subject for gating diagnostic imaging and therapeutic delivery applications which require respiration phase and/or respiration amplitude gating.

It should be understood that one of skilled in this art would readily understand various aspects of image processing, and methods for generating time-series signals from pixels obtained from batches of image frames in a video. Such methods are disclosed in several of the incorporated references by Lalit K. Mestha, Edgar Bernal, Beilei Xu and Survi Kyal. One skilled in this art would also readily understand various signal processing techniques including methods for uncovering independent source signal components from a set of observations that are composed of linear mixtures of underlying sources. Such methods are taught in: “Independent Component Analysis”, Wiley (2001), ISBN-13: 978-0471405405, and “Blind Source Separation: Theory and Applications”, Wiley (2014), ISBN-13: 978-1118679845, which are incorporated herein in their entirety by reference. One skilled in this art would also have a working knowledge of algorithms involving multivariate analysis and linear algebra as are needed to effectuate non-negative matrix factorizations. Such techniques are taught in: “Nonnegative Matrix and Tensor Factorizations: Applications to Exploratory Multi-Way Data Analysis and Blind Source Separation”, Wiley (2009), ISBN-13: 978-0470746660, which is incorporated herein in its entirety by reference.

### Non-Limiting Definitions

“Respiratory function”, as is normally understood, involves inhaling and exhaling a volume of air in/out of the lungs. The expansion and contraction of the lungs and chest walls during respiration induces a movement in the subject's body which is captured in a video of the subject.

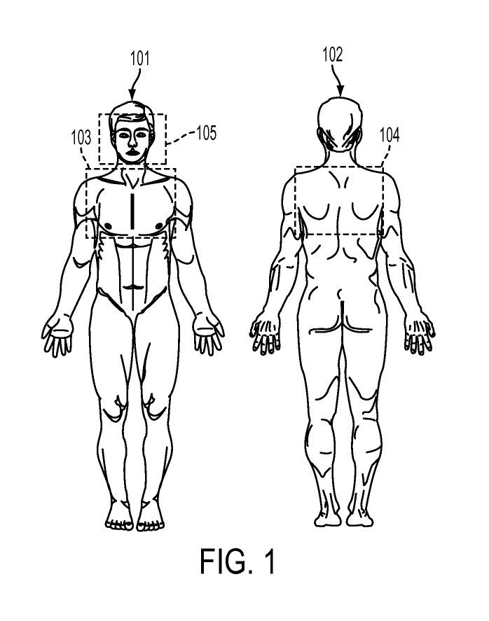

A “subject' refers to a living being with a respiratory function. Although the term “person” or “patient” may be used throughout this disclosure, it should be appreciated that the subject may be something other than a human such as, for example, a primate. Therefore, the use of such terms is not to be viewed as limiting the scope of the appended claims strictly to human beings with a respiratory function. FIG. 1 shows an anterior (frontal) view 101 of an adult human as well as a posterior (rear) view 102 where plethysmographic signals corresponding to respiratory function can be acquired by a video imaging device. Signals associated with respiratory function can be acquired by the video imaging device in the subject's anterior thoracic region 103, the posterior thoracic region 104, and the facial region 105. Such signals can also be acquired from the left and right sides of the thoracic cage (not shown) including from the abdominal area.

A “video” refers to a plurality of time-sequential image frames captured of one or more regions of interest of a subject where a signal corresponding to the subject's respiratory function can be registered by at least one imaging channel of the video imaging device used to acquire that video. The video may also contain other components such as, audio, time, date, reference signals, frame information, and the like. The video may be processed to compensate for motion induced blur, imaging blur, or slow illuminant variation. The video may also be processed to enhance contrast or brightness. Independent region selection can be used to emphasize certain content in the video such as, for example, a region containing an area of exposed skin. If camera related noise or environmental factors are adversely affecting extraction of the time-series signals from the image frames of the video, compensation can be effectuated using the methods disclosed in the incorporated reference: “Removing Environment Factors From Signals Generated From Video Images Captured For Biomedical Measurements”, Lalit K. Mestha et al. A user may select a subset of the image frames of the video for processing. The video of the subject is captured or is otherwise acquired by a video imaging device.

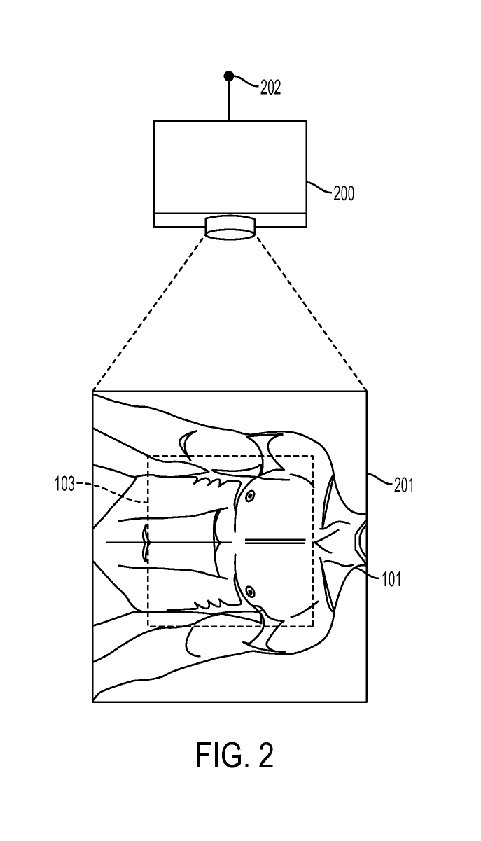

A “video imaging device” refers to a single-channel or multi-channel video camera for capturing or acquiring video of the subject. Video imaging devices include: a color video camera, a monochrome video camera, an infrared video camera, a multispectral video imaging device, a hyperspectral video camera, or a hybrid device comprising any combination hereof. The video imaging device may be a webcam. FIG. 2 shows an example video imaging device 200 capturing image frames (individually at 201) of a region of interest 103 of the subject 101. The video imaging device has a communication element 202 (shown as an antenna) which effectuates a bi-directional communication with a remote device such as a computer workstation over a wireless network where the image frames are received for processing in accordance with the methods disclosed herein. The video camera comprises one or more lens which function to focus received reflected light. Focused and filtered light is directed on to one or more photodetectors which independently record intensity values at pixel locations along a multi-dimensional grid. The received light is spatially resolved to form the image. If the video imaging device used to capture the video of the subject is a color video camera with red, green and blue (RGB) channels, intensity components can be obtained from any or a combination of the imaging channels on a per-pixel basis. The video imaging device may incorporate memory, a storage device, and a video analysis module comprising one or more microprocessors for executing machine readable program instructions for analyzing the received video in accordance with the teachings hereof. Such a video analysis module may comprise, in whole or in part, a software application working alone or in conjunction with one or more hardware resources. The received video is processed to isolate one or more regions of interest of the subject.

A “region of interest” refers to at least a partial view of the subject as seen through the aperture of the video imaging device where a signal corresponding to respiratory function can be registered by at least one imaging channel of the video imaging device used to capture that video. A region of interest may be an area of exposed skin or an area covered by a sheet or an article of clothing. Body regions which move during a respiratory cycle include the thoracic regions 103 and 104, and the facial region 105. Regions of interest can be identified in image frames automatically using a variety of techniques known in the arts including: pixel classification, object identification, facial recognition, color, texture, spatial features, spectral information, and pattern recognition. One or more regions of interest may be manually identified by a user input or selection. For example, during system setup and configuration, an operator or technician may use a mouse or a touchscreen display to manually draw a rubber-band box around one or more areas in an image frame of the subject displayed on a monitor or display device thereby defining a region of interest. Pixels in the region(s) of interest are isolated in the image frames of the video for processing.

“Isolating pixels” in a region of interest can be effectuated using any of a wide array of techniques that are well established in the image processing arts which include: pixel classification based on color, texture, spatial features, and the like. Pixels isolate in the region of interest may be weighted, averaged, normalized, or discarded, as needed. Pixels may be grouped for processing. Pixels may be spatially filtered or amplitude filtered to reduce noise. A time-series signal is generated from values of pixels or from values of groups of pixels isolated in a region of interest.

A “time-series signal” is a signal that contains frequency components that relate to motion due to respiratory function. Time-series signals are generated from pixels isolated in a region of interest in a temporal direction across a desired set of time-sequential image frames in the video. Methods for generating time-series signals from video are disclosed in several of the incorporated references by Lalit K. Mestha, Edgar Bernal, Beilei Xu and Survi Kyal. Some or all of the time-series signals may be weighted. Time-series signals may be normalized and filtered to remove undesirable frequencies. For example, a filter with a low cutoff frequency f_{L} and a high cutoff frequency f_{H}, where f_{L} and f_{H} are a function of the subject's tidal breathing rate, may be used to filter the signals. The cutoff frequencies may be a function of the subject's respiratory health, age, and medical history. Features are extracted from the time-series signals and formed into vectors.

A “feature vector” contains features extracted from the time-series signals. Methods for generating vectors from individual elements are well understood in the mathematical arts. In one embodiment, the features are coefficients of a quadratic polynomial fit to one or more segments of the time-series signal. Features extracted from the time-series signals may be eigen features, coefficients of a filter, coefficients of a discrete cosine transform of the signal, coefficients of a wavelet transform of the signal, a standard deviation of the signal, root mean square values of the signal, a norm of the signal, signal values at end-inspiration and end-expiration point, an interval between these points, pixel intensity values, pixel location in the image frame, time/reference data, and motion component information such as amount of pixel movement between adjacent frames. Other features may be obtained from deep learning algorithms. Pixels may be grouped and their mean, median, standard deviation, or higher order statistics computed and added to a respective feature vector. Values can be aggregated and added as features such as, for instance, an algebraic sum of pixel values obtained from each of the imaging channels of the video imaging device used to acquire the video. Feature vectors are clustered.

A “cluster” contains one or more features extracted from the time-series signals Clusters are formed using, for example, K-means testing, vector quantization (such as the Linde-Buzo-Gray algorithm), constrained clustering, fuzzy clustering, nearest neighbor clustering, linear discriminant analysis, Gaussian Mixture Model, and a support vector machine. Various thresholds may be employed to facilitate discrimination amongst features for clustering purposes. Clusters may be labeled based on apriori knowledge such as, for example, respiratory conditions, respiratory-related events, medical histories, and the like. Clusters may be formed manually or automatically. The clustering may be unsupervised.

A “representative signal” is obtained for each cluster by averaging, in a temporal direction, the time-series signals corresponding to pixels represented by the feature vectors in that clusters. Methods for averaging signals together and for obtaining a signal from a plurality of signals are well established in the mathematical and signal processing arts. Once a representative signal has been obtained for each cluster, one of the clusters is selected.

“Selecting a cluster” means to manually or automatically identify one cluster from the plurality of clusters. In one embodiment, cluster selection involves a spectral compaction approach. In another embodiment, cluster selection is based on a distance metric such as Euclidean, Mahalanobis, Bhattacharyya, Hamming, or Hellinger distance. Distances can be with respect to a known reference signal representing the breathing pattern of the subject or from some other metric such as, for example, determined in relation to a center of the cluster, a boundary element of the cluster, and the like. Distances may comprise a weighted sum of some or all of the features in a given cluster. Selection of a cluster can be made by a user making a selection via a mouse or keyboard. In one embodiment, the selected cluster's associated representative signal is used to obtain the respiration gating signal. In another embodiment, the selected cluster's associated representative signal is used to identify a respiratory pattern for the subject and the respiration gating signal is obtained from a signal associated with the identified respiratory pattern.

A “respiratory pattern” refers to a pattern of the subject's breathing, as is understood in the medical arts. Breathing patterns include Eupnea, Bradypnea, Tachypnea, Hypopnea, Apnea, Kussmaul, Cheyne-Stokes, Biot's, Ataxic, Apneustic, Agonal, and Thoracoabdominal. Methods for obtaining a respiratory pattern are disclosed in the incorporated references by Prathosh A. Prasad and Lalit K. Mestha.

A “respiration gating signal” is a signal used to gate (i.e., trigger) data acquisition of any of a variety of diagnostic imaging devices and therapeutic delivery applications (individually a “device”) which require gating to coincide with patient motion due to respiratory function.

A “device” which is intended to be gated (“triggered”) by the respiration gating signal generated using the methods disclosed herein can be any diagnostic imaging and therapeutic delivery application including: Dual Energy Radiography, Computed Tomography (CT), Tomographic Synthesis in Mammography, Magnetic Resonance Imaging (MRI), Positron Emission Tomography (PET), PET-CT, and PET-MRI. This list is intended to be illustrative and not limiting. As such, presently unforeseen imaging devices and therapeutic delivery applications which utilize the present respiration gating signal are intended to fall within the scope of the appended claims.

“Receiving a video of a subject” is intended to be widely construed and includes retrieving, capturing, acquiring, or otherwise obtaining video image frames for processing. The video can be received or retrieved from a remote device over a network, or from a media such as a CDROM or DVD. The video can be received directly from a memory or storage device of the video imaging device used to capture or acquire that video. Video may be downloaded from a web-based system or application which makes video available for processing. Video can also be received from an application such as those which are available for handheld cellular devices and processed on the cellphone or other handheld computing device such as an iPad or Tablet-PC.

It should be appreciated that the recited steps of: “receiving”, “generating”, “extracting”, “forming”, “clustering”, “averaging”, “selecting”, “using”, “determining”, “performing”, “weighting”, “filtering”, “detrending”, “upsampling”, “down-sampling”, “smoothing”, “transforming”, “synchronizing”, “communicating”, “grouping”, associating”, “processing”, and the like, include the application of any of a variety of signal processing techniques as are known in the signal processing arts, as well as mathematical operations according to any specific context or for any specific purpose. It should be appreciated that such steps may be facilitated or otherwise effectuated by a microprocessor executing machine readable program instructions such that an intended functionality can be effectively performed.

### Example Flow Diagram

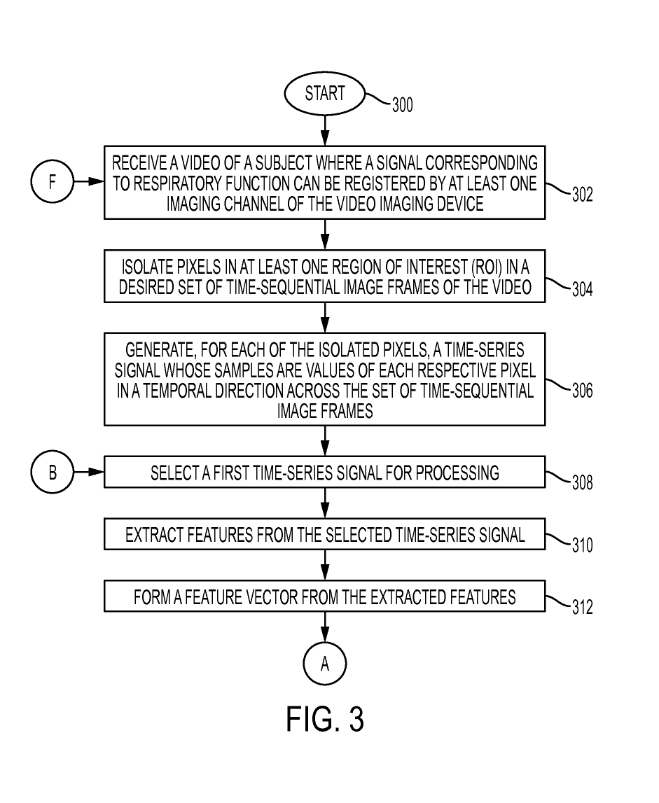

Reference is now being made to the flow diagram of FIG. 3 which illustrates one embodiment of the present method for generating a respiration gating signal from a video of a subject. Flow processing begins at step 300 and immediately proceeds to step 302.

At step 302, receiving a video of a subject where a signal corresponding to respiratory function can be registered by at least one imaging channel of a video imaging device.

At step 304, isolate pixels in at least one region of interest in a desired set of time-sequential image frames of the video.

At step 306, generate, for each of the isolated pixels, a time-series signal whose samples are values of each respective pixels in a temporal direction across the time-sequential image frames.

At step 308, select a first time-series signal for processing.

At step 310, extract features from the selected time-series signal.

At step 312, form a feature vector from the extracted features.

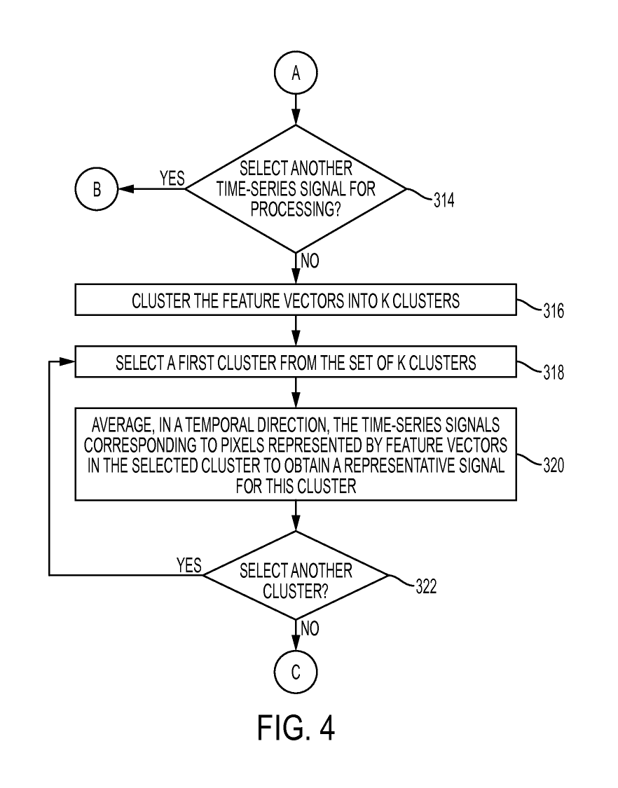

Reference is now being made to the flow diagram of FIG. 4 which is a continuation of the flow diagram of FIG. 3 with flow processing continuing with respect to node A.

At step 314, a determination is made whether any more time-series signals are to be processed. If so then processing repeats with respect to node B wherein, at step 308, a next time-series signal is selected or otherwise identified for processing. Features are extracted from this next time-series signal and formed into a feature vector. Processing repeats in a similar manner until no more time-series signals remain to be selected.

At step 316, cluster the feature vectors into K clusters. In one embodiment, K=6 clusters.

At step 318, select a first cluster from the set of clusters.

At step 320, average, in a temporal direction, the time-series signals corresponding to pixels represented by feature vectors in the selected clusters to obtain a representative signal for this cluster.

At step 322, a determination is made whether more clusters remain to be selected. If so then processing repeats with respect to step 318 wherein a next cluster is selected or is otherwise identified from the set of clusters for processing. All the time-series signals corresponding to pixels represented by the feature vectors in this next selected cluster are averaged to obtain a representative signal for this cluster. Processing repeats in a similar manner until no more clusters remain to be processed.

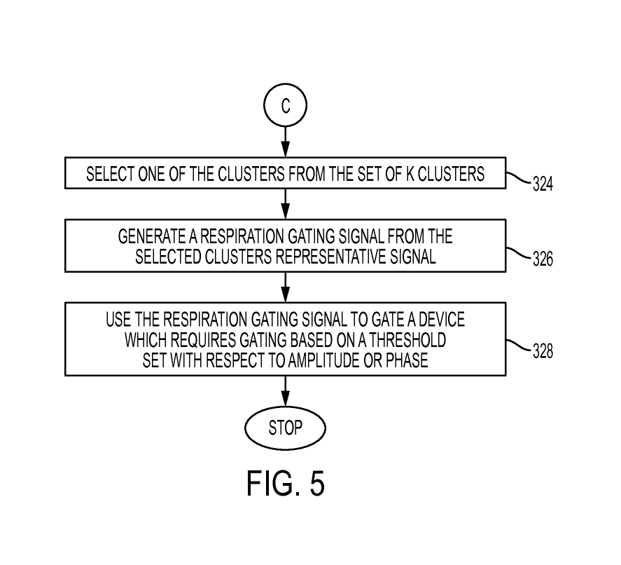

Reference is now being made to the flow diagram of FIG. 5 which is a continuation of the flow diagram of FIG. 4 with flow processing continuing with respect to node C.

At step 324, select one of the clusters from the set of K clusters. This selection can be based on a user selection, a distance metric, or a spectral compaction method.

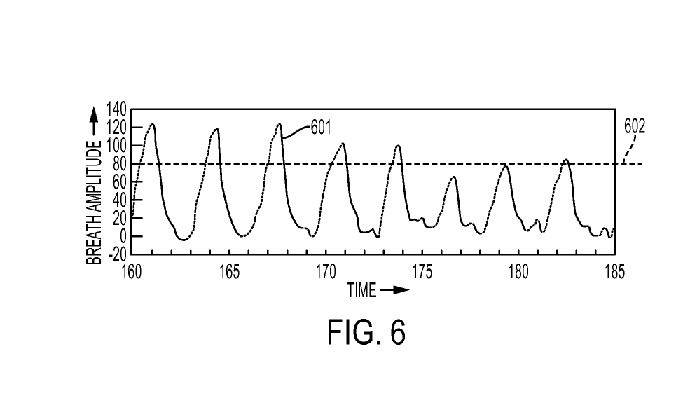

At step 326, generate a respiration gating signal from the selected cluster's representative signal. FIG. 6 shows an example respiration gating signal 601 with a threshold 602 set with respect to respiration amplitude. The threshold may be manually or automatically set. In another embodiment, the selected cluster's representative signal is used to identify a breathing pattern and the respiration gating signal is obtained from a signal associated with the identified breathing pattern.

At step 328, use the respiration gating signal to gate a device which requires gating based on a threshold set with respect to any of: respiration phase and respiration amplitude. Thereafter, in this embodiment, further processing stops.

It should be understood that the flow diagrams depicted herein are illustrative. One or more of the operations illustrated in the flow diagrams may be performed in a differing order. Other operations may be added, modified, enhanced, or consolidated. Variations thereof are intended to fall within the scope of the appended claims. All or portions of the flow diagrams may be implemented partially or fully in hardware in conjunction with machine executable instructions.

### Example Video Processing System

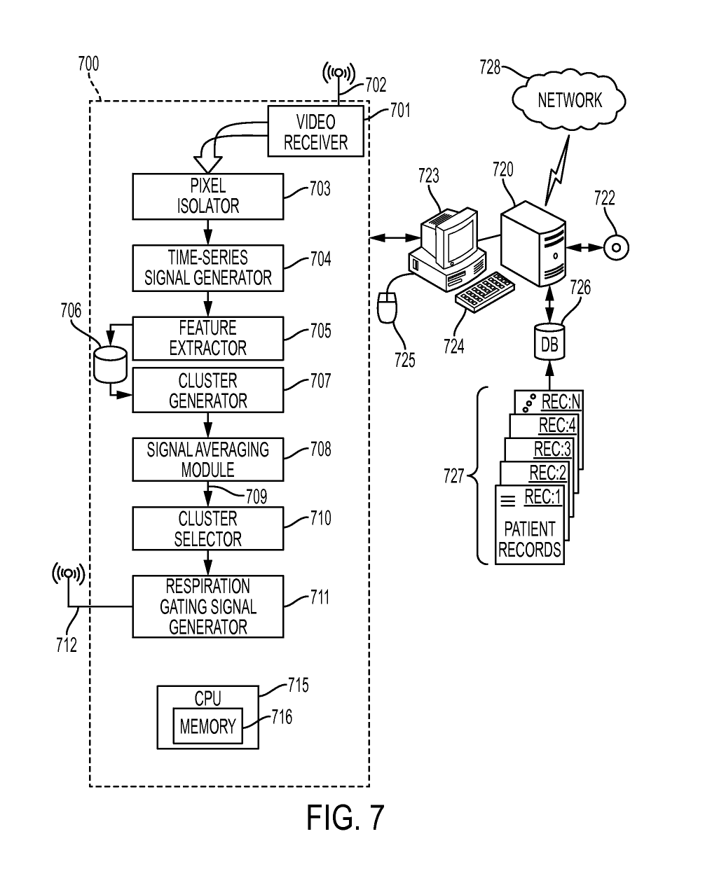

Reference is now being made to FIG. 7 which shows a block diagram of one example video processing system 700 for processing a video in accordance with the embodiments described with respect to the flow diagrams of FIGS. 3-5. Reference is now being made to FIG. 7 which shows a block diagram of one example video processing system 700 for processing a video in accordance with the embodiments described with respect to the flow diagrams of FIGS. 3-5.

Video Receiver 701 wirelessly receives the video via antenna 702 having been transmitted thereto from the video imaging device 200 of FIG. 2. Pixel Isolator Module 703 processes the received video and proceeds to isolate pixels in the region of interest. Time-Series Signal Generator 704 generates a time-series signal for each of the isolated pixels or, alternatively, for groups of isolated pixels, in a temporal direction across a defined duration of time-sequential image frames. Feature Extractor Module 705 receives the time-series signals and extracts features from those signals. The extracted features are formed into feature vectors and stored to storage device 706. Cluster Generator 707 retrieves the feature vectors from the storage device and sorts the feature vectors into K clusters. Signal Averaging Module 708 averages the time-series signals associated with each of the feature vectors in each of the clusters to obtain a representative signal (collectively at 709) for each cluster. Cluster Selector 710 automatically selects one of the clusters based on a distance metric. Alternatively, a user manually selects one of the clusters using the computer workstation. Respiration Gating Signal Generator 711 receives the selected cluster's corresponding associated representative signal and generates a respiration gating signal with respect to a pre-defined threshold set by a user. The threshold may be retrieved from storage. Other aspects which may be used to determine threshold or which may be used to modify the gating signal can be retrieved from storage device 706 using a communication pathway (not shown). The generated respiration gating signal is communicated wirelessly to a diagnostic imaging and therapeutic delivery application via communication element 712, shown as an antenna. The respiration gating signal can also be communicated to one or more devices over network 728. The gating thresholds can be dynamically adjusted by the user using the workstation 720. The user may further manipulate the generated respiration gating signal as desired or as is otherwise needed to effectuate control of a device intended to be gated hereby.

Central Processing Unit 715 retrieves machine readable program instructions from a memory 716 and is provided to facilitate the functionality of any of the modules of the system 700. CPU 715, operating alone or in conjunction with other processors, may be configured to assist or otherwise perform the functionality of any of the modules or processing units of the system 700 as well as facilitating communication between the system 700 and the workstation 720.

Workstation 720 has a computer case which houses various components such as a motherboard with a processor and memory, a network card, a video card, a hard drive capable of reading/writing to machine readable media 722 such as a floppy disk, optical disk, CD-ROM, DVD, magnetic tape, and the like, and other software and hardware as is needed to perform the functionality of a computer workstation. The workstation includes a display device 723, such as a CRT, LCD, or touchscreen display, for displaying information, regions of interest, video image frames, clusters, distances, feature vectors, computed values, thresholds, medical information, test results, and the like, which are produced or are otherwise generated by any of the modules or processing units of the video processing system. A user can view any such information and make a selection from various menu options displayed thereon. Keyboard 724 and mouse 725 effectuate a user input or selection.

It should be appreciated that the workstation 720 has an operating system and other specialized software configured to display alphanumeric values, menus, scroll bars, dials, slideable bars, pull-down options, selectable buttons, and the like, for entering, selecting, modifying, and accepting information needed for performing various aspects of the methods disclosed herein. A user may use the workstation to identify a set of image frames of interest, define features, select clusters, set various parameters, and otherwise facilitate the functionality of any of the modules or processing units of the video processing system. A user or technician may utilize the workstation to modify, add or delete any of the feature vectors as is deemed appropriate. A user or technician may utilize the workstation to further define clusters, add clusters, delete clusters, combine clusters and move feature vectors to various clusters as is deemed appropriate. The user may adjust various parameters being utilized or dynamically adjust in real-time, system or threshold settings or any parameters of the video imaging device used to capture the video. User inputs and selections may be stored/retrieved in any of the storage devices 706, 722 and 726. Default settings and initial parameters can be retrieved from any of the storage devices. Although shown as a desktop computer, it should be appreciated that the workstation can be a laptop, mainframe, tablet, notebook, smartphone, or a special purpose computer such as an ASIC, or the like. The embodiment of the workstation is illustrative and may include other functionality known in the arts.

The workstation implements a database in storage device 726 wherein records are stored, manipulated, and retrieved in response to a query. Such records, in various embodiments, take the form of patient medical histories stored in association with information identifying the patient (collectively at 727). It should be appreciated that the database may be the same as storage device 706 or, if separate devices, may contain some or all of the information contained in any of the storage devices shown. Although the database is shown as an external device, the database may be internal to the workstation mounted, for example, on a hard drive within the computer case.

Any of the components of the workstation may be placed in communication with any of the modules of the video processing system 700 or any devices placed in communication therewith. Moreover, any of the modules of the video processing system can be placed in communication with storage device 726 and/or computer readable media 722 and may store/retrieve therefrom data, variables, records, parameters, functions, and/or machine readable/executable program instructions, as needed to perform their intended functionality. Further, any of the modules or processing units of the video processing system may be placed in communication with one or more remote devices over network 728.

It should be appreciated that some or all of the functionality performed by any of the modules or processing units of the video processing system 700 can be performed, in whole or in part, by the workstation. The embodiment shown is illustrative and should not be viewed as limiting the scope of the appended claims strictly to that configuration. Various modules may designate one or more components which may, in turn, comprise software and/or hardware designed to perform the intended function.

### Various Embodiments

The teachings hereof can be implemented in hardware or software using any known or later developed systems, structures, devices, and/or software by those skilled in the applicable arts without undue experimentation from the functional description provided herein with a general knowledge of the relevant arts. Software applications may be executed by processors on different hardware platforms or emulated in a virtual environment and may leverage off-the-shelf software. One or more aspects of the methods described herein are intended to be incorporated in an article of manufacture. The article of manufacture may be shipped, sold, leased, or otherwise provided separately either alone or as part of a product suite or a service. The above-disclosed and other features and functions, or alternatives thereof, may be desirably combined into other different systems or applications. Presently unforeseen or unanticipated alternatives, modifications, variations, or improvements may become apparent and/or subsequently made by those skilled in this art which are also intended to be encompassed by the following claims.

The teachings of any publications referenced herein are incorporated in their entirety by reference having been made thereto.

What is claimed is:

1. A method for generating a respiration gating signal from a video of a subject for gating diagnostic imaging and therapeutic delivery applications which require respiration phase and/or respiration amplitude gating, the method comprising:
receiving a video of a subject, said video comprising N≧2 image frames of a region of interest of said subject where a signal corresponding to the subject's respiratory function can be registered by at least one imaging channel of a video imaging device used to capture said video, said region of interest comprising P pixels, where P≧2;
generating a plurality of time-series signals [S₁, . . . , S_{P}} each of duration N whose samples are values of pixels in said region of interest in said image frames;
extracting, for each of said time-series signals, a set of features and forming a P-number of M-dimensional feature vectors, where M≧2;
clustering said feature vectors into K≧2 clusters;
averaging, in a temporal direction, all time-series signals corresponding to pixels represented by said feature vectors in each of said clusters to obtain a representative signal for each cluster;
selecting one of said clusters;
generating a respiration gating signal from one of:
(A) said selected cluster's representative signal; and
(B) a respiratory pattern associated with said selected cluster's representative signal; and
using said respiration gating signal to gate a device which requires gating based on a threshold set with respect to any of: respiration phase and respiration amplitude.

2. The method of claim 1, wherein said video imaging device is any of: a color video camera, an infrared video camera, a monochrome video camera, a multispectral video imaging device, a hyperspectral video camera, a webcam, and a hybrid device comprising any combination hereof.

3. The method of claim 1, wherein said device is a diagnostic imaging device and therapeutic delivery application comprising of any of: Dual Energy Radiography, Computed Tomography (CT), Tomographic Synthesis in Mammography, Magnetic Resonance Imaging (MRI), Positron Emission Tomography (PET), PET-CT, and PET-MRI.

4. The method of claim 1, wherein video camera captures images from any of: posterior, anterior, abdominal, and thoracic regions of the body.

5. The method of claim 1, wherein each pixel in said region of interest has an associated time-series signal.

6. The method of claim 1, further comprising grouping pixels in said region of interest and generating a time-series signal for each of said pixel groups.

7. The method of claim 6, wherein, in advance of generating time-series signals for said groups of pixels, further comprising any of:
spatial filtering said groups of pixels; and
amplitude filtering pixels in said groups.

8. The method of claim 1, wherein said time-series signals are divided into at least two batches of smaller time-series signals in a temporal direction.

9. The method of claim 1, wherein said features comprise any of: coefficients of a quadratic polynomial fit to at least a portion of said time-series signal, eigen features, coefficients of a filter, coefficients of a discrete cosine transform, and coefficients of a wavelet transform.

10. The method of claim 1, wherein each of said feature vectors individually quantifies an overall temporal orientation of a respective time-series signal, said feature vectors being clustered according to their temporal alignment.

11. The method of claim 1, wherein, in advance of extracting said features, processing said time-series signals comprising any of:
weighting at least a segment of one of said time-series signals;
band pass filtering any of said time-series signals to restrict frequencies of interest;
filtering any of said time-series signals to remove unwanted artifacts;
detrending said time-series signals to remove low frequency and non-stationary components;
averaging any of said time-series signals to obtain a composite signal;
discarding at least a portion of any of said time-series signals;
upsampling any of said time-series signals to a standard sampling frequency;
down-sampling any of said time-series signals;
smoothing at least a segment of any of said time-series signals;
transforming any of said time-series signals into an alternate domain; and
synchronizing any of said time-series signals with respect to time.

12. The method of claim 1, wherein clustering said features into K clusters comprises at least one of: K-means testing, vector quantization, constrained clustering, fuzzy clustering, linear discriminant analysis, a Gaussian Mixture Model, nearest neighbor clustering, manual sorting, and a support vector machine.

13. The method of claim 1, wherein said cluster selection is based on a distance metric comprising any of: Euclidean, Mahalanobis, Bhattacharyya, Hamming, and a Hellinger distance determined in relation to any of: a center of said cluster, a boundary element of said cluster, and a weighted sum of at least some elements in said cluster.

14. The method of claim 1, wherein said gating is any of: time-synchronized and time-delayed.

15. The method of claim 1, further comprising communicating said respiration gating signal to any of: a memory, a storage device, a smartwatch, a smartphone, a display, an iPad, a tablet-PC, a laptop, a workstation, and a remote device over a network.

16. A system for generating a respiration gating signal from a video of a subject for gating diagnostic imaging and therapeutic delivery applications which require respiration phase and/or respiration amplitude gating, the system comprising:
a storage device; and
a processor in communication with said storage device, said processor executing machine readable instructions for:
receiving a video of a subject, said video comprising N≧2 image frames of a region of interest of said subject where a signal corresponding to the subject's respiratory function can be registered by at least one imaging channel of a video imaging device used to capture said video, said region of interest comprising P pixels, where P≧2;
generating a plurality of time-series signals {S₁, . . . , S_{P}} each of duration N whose samples are values of pixels in said region of interest in said image frames;
extracting, for each of said time-series signals, a set of features and forming a P-number of M-dimensional feature vectors, where M≧2;
clustering said feature vectors into K≧2 clusters;
averaging, in a temporal direction, all time-series signals corresponding to pixels represented by said feature vectors in each of said clusters to obtain a representative signal for each cluster;
selecting one of said clusters;
generating a respiration gating signal from one of:
(A) said selected cluster's representative signal; and
(B) a respiratory pattern associated with said selected cluster's representative signal; and
using said respiration gating signal to gate a device which requires gating based on a threshold set with respect to any of: respiration phase and respiration amplitude.

17. The system of claim 16, wherein said video imaging device is any of: a color video camera, an infrared video camera, a monochrome video camera, a multispectral video imaging device, a hyperspectral video camera, a webcam, and a hybrid device comprising any combination hereof.

18. The system of claim 16, wherein said device is a diagnostic imaging device and therapeutic delivery application comprising of any of: Dual Energy Radiography, Computed Tomography (CT), Tomographic Synthesis in Mammography, Magnetic Resonance Imaging (MRI), Positron Emission Tomography (PET), PET-CT, and PET-MRI.

19. The system of claim 16, wherein video camera captures images from any of: posterior, anterior, abdominal, and thoracic regions of the body.

20. The system of claim 16, wherein each pixel in said region of interest has an associated time-series signal.

21. The system of claim 16, further comprising grouping pixels in said region of interest and generating a time-series signal for each of said pixel groups.

22. The system of claim 21, wherein, in advance of generating time-series signals for said groups of pixels, further comprising any of:
spatial filtering said groups of pixels; and
amplitude filtering pixels in said groups.

23. The system of claim 16, wherein said time-series signals are divided into at least two batches of smaller time-series signals in a temporal direction.

24. The system of claim 16, wherein said features comprise any of: coefficients of a quadratic polynomial fit to at least a portion of said time-series signal, eigen features, coefficients of a filter, coefficients of a discrete cosine transform, and coefficients of a wavelet transform.

25. The system of claim 16, wherein each of said feature vectors individually quantifies an overall temporal orientation of a respective time-series signal, said feature vectors being clustered according to their temporal alignment.

26. The system of claim 16, wherein, in advance of extracting said features, processing said time-series signals comprising any of:
weighting at least a segment of one of said time-series signals;
band pass filtering any of said time-series signals to restrict frequencies of interest;
filtering any of said time-series signals to remove unwanted artifacts;
detrending said time-series signals to remove low frequency and non-stationary components;
averaging any of said time-series signals to obtain a composite signal;
discarding at least a portion of any of said time-series signals;
upsampling any of said time-series signals to a standard sampling frequency;
down-sampling any of said time-series signals;
smoothing at least a segment of any of said time-series signals;
transforming any of said time-series signals into an alternate domain; and
synchronizing any of said time-series signals with respect to time.

27. The system of claim 16, wherein clustering said features into K clusters comprises at least one of: K-means testing, vector quantization, constrained clustering, fuzzy clustering, linear discriminant analysis, a Gaussian Mixture Model, nearest neighbor clustering, manual sorting, and a support vector machine.

28. The system of claim 16, wherein said cluster selection is based on a distance metric comprising any of: Euclidean, Mahalanobis, Bhattacharyya, Hamming, and a Hellinger distance determined in relation to any of: a center of said cluster, a boundary element of said cluster, and a weighted sum of at least some elements in said cluster.

29. The system of claim 16, wherein said gating is any of: time-synchronized and time-delayed.

30. The system of claim 16, further comprising communicating said respiration gating signal to any of: a memory, a storage device, a smartwatch, a smartphone, a display, an iPad, a tablet-PC, a laptop, a workstation, and a remote device over a network.
